(19) United States
(12) Patent Application Publication
(10) Pub. No.: US 2009/0226951 A1
(43) Pub. Date: Sep. 10, 2009

(54) Analysis method of mixed liquid in waste water treatment

(76) Inventor: Takao Ogawa, Kanagawa (JP)

Correspondence Address: BIRCH STEWART KOLASCH & BIRCH, PO BOX 747, FALLS CHURCH, VA, 22040-0747, US

(21) Appl. No.: 11/886,242

(22) PCT Filed: Jun. 16, 2006
(86) PCT No.: PCT/JP06/12112

§ 371 Date: Sep. 13, 2007

(51) Int. Cl. C12Q 1/02

(52) U.S. Cl. 435/29

(57) ABSTRACT

[object] A technique for analyzing an oxygen consumption and an oxygen consumption rate in a process in which components of waste water are decomposed by aerobic microbes is provided.

[Solving means] An oxygen consumption rate kₓ=KLa·(DOhf−highDOₓ) of a last block (block x) containing only one BOD component is first obtained by using the above approximate expression of a dissolved oxygen-concentration change curve, Δtₓ=tₓ−tₓ₋₁, and BODₓ=kₓ·Δtₓ; kₓ₋₁ of a block which contains two BOD components and which is second to the last block is then obtained from kₓ₋₁=KLa·(DOhf−highDOₓ₋₁)−kₓ=KLa·(highDOₓ−highDOₓ₋₁); by sequentially performing this calculation to a first block, an oxygen consumption rate kᵢ of each BOD component is obtained by using the relationship represented by kᵢ=KLa·(highDOᵢ₊₁−highDOᵢ); and in addition, the BOD concentration of each BOD component is also obtained based on pBODᵢ=kᵢ·tₓ.

### TECHNICAL FIELD

The present invention relates to an analysis method in a waste water treatment by an activated sludge model, and more particularly, relates to an analysis method of a mixed liquid in a process in which components of waste water are decomposed by aerobic microbes.

### BACKGROUND ART

In a waste water treatment using aerobic microbes, when a mixed liquid containing activated sludge and waste water is aerated by an aeration apparatus, the change in dissolved oxygen concentration DO in the mixed liquid is represented by the following equation.

$$\begin{matrix}[ Formula 2 ] & \\ \frac{d DO}{d t} = KLa ( DOsat - DO ) - ( ASact + BODact ) & Equation ( 1 )\end{matrix}$$

In the above equation, DOsat indicates a saturated dissolved oxygen concentration [mg/l], DO indicates a dissolved oxygen concentration [mg/l] in an aeration tank, KLa indicates a total mass transfer coefficient [l/min] when the difference between the saturated dissolved oxygen concentration of the mixed liquid and the dissolved oxygen concentration thereof at that time is regarded as a driving force, ASact indicates an oxygen consumption rate [mg/l/min] used by the activated sludge for respiration, and BODact indicates an oxygen consumption rate [mg/l/min] used by the activated sludge for decomposition of BOD components. The first term of the right side of the equation (1) indicates an oxygen supply rate from the aeration apparatus, and the second term indicates an oxygen consumption rate used by respiration of the activated sludge and decomposition of BOD.

Since BODact varies, Equation (1) cannot be simply integrated; however, since ASact indicates the oxygen consumption rate by respiration of microbes, in the range of DO>0.5 mg/l during a measurement time, ASact can be regarded as approximately constant, and in the state in which BODact≅0 holds, the following equation (2) holds and can be integrated.

$$\begin{matrix}[ Formula 3 ] & \\ \frac{d DO}{d t} = KLa ( DOsat - DO ) - ASact & Equation ( 2 )\end{matrix}$$

When the mixed liquid is aerated in the aeration apparatus for a sufficiently long time so that BOD≅0 mg/l holds in the mixed liquid, and a dissolved oxygen concentration at a time at which it reaches an approximately constant value is represented by DOhf, Equation (2) can be integrated, so that Equation (3) of

DO=DOhf−(DOhf−DO₀)exp(−KLa·t)

holds. Where DOhf=DOsat−ASact/KLa. The change of DO in Equation (3) is represented by a curved line such as A shown in FIG. 1.

Now, a DO change curve is shown by a solid line B in FIG. 1 which is obtained when waste water to be measured is added in the state in which DO is an initial value DO₀, followed by aeration, by using a mixed liquid in which aeration is sufficiently performed beforehand so that BOD≅0 mg/l holds, and KLa and DOhf are measured beforehand. By addition of the waste water to be measured, BOD components are present in the mixed liquid, and the value of BODact is changed from a large value to a small value with the aeration time primarily because of the change in BOD component to be decomposed. When a BOD component to be finally decomposed disappears, BODact becomes substantially 0. Consequently, although Equation (1) cannot be easily integrated as compared to Equation (3), the change in DO is represented by a curved as shown by B in FIG. 1. That is, during the decomposition, DO is changed at a low level at which the oxygen supply rate and the oxygen consumption rate (ASact+BODact) balance with each other, and when the decomposition is complete, DO increases and then reaches a constant value DOhf.

Furthermore, the applicant of the present invention disclosed a method for analyzing a dissolved oxygen change curve when a plurality of BOD components is contained in a mixed liquid (see Patent Document 1). With reference to FIG. 2, the outline of the method will be described. When the curve showing the change in dissolved oxygen concentration, which is obtained by addition of waste water to be measured, is divided into blocks, a dissolved oxygen concentration at the start in a block 2 is represented by DO1, and the start time of that block is represented by t1, an imaginary dissolved oxygen-concentration change curve A1 calculated by Equation (4) of

DO=DOhf−(DOhf−DO₁)exp(−KLa·(t−t₁))

indicates the change in dissolved oxygen concentration from the time t1 of this block to a time at which a mixed liquid is aerated in which BOD is substantially 0 mg/l. In addition, an imaginary dissolved oxygen-concentration change curve A2 obtained by Equation (5) of

DO=DOhf−(DOhf−DO₂)exp(−KLa·(t−t₂))

indicates the change in dissolved oxygen concentration obtained when the mixed liquid is aerated so that BOD which is decomposable in the block becomes 0. Accordingly, in FIG. 2, a value obtained by multiplying KLa and an area S₂ surrounded by the dissolved oxygen-concentration change curve in this block, the imaginary dissolved oxygen-concentration change curve A1, and the imaginary dissolved oxygen-concentration change curve A2 is an oxygen consumption used by microbes for decomposition of BOD components in this block, that is, the BOD value. The relationship described above holds for n in the range of 1 to 4 shown in FIG. 2.

Since BODact varies during a long aeration process, Equation (1) cannot be simply integrated; however, in the individual divided blocks, different enzymes and microbes decompose respective materials, and hence BODact can be regarded as constant in each range. When this value is represented by BODactn, the solution of Equation (1) can be easily obtained so that Equation 6 of

DO=highDOₙ−(highDOₙ−DOₙ₋₁)exp(−KLa·(t−tₙ₋₁))

holds. Where highDOₙ indicates DOsat−(ASact+BODactn)/KLa. In the above equation, DOₙ indicates a dissolved oxygen concentration in an n-th block, DOₙ₋₁ indicates a DO value at the start of the block, t indicates an aeration time, and tₙ₋₁ indicates a start time of the block. In addition, highDOₙ indicates a DO value in the block at which the oxygen supply rate by aeration balances with the oxygen consumption rate by respiration of microbes and by decomposition of a BOD component. As shown in FIG. 2, highDOₙ in a block 1 is highDO₁ which exhibits first a curved line and then a straight line; however, in a block 3, since subsequent decomposition starts before highDOₙ exhibits a perfect straight line, highDO₃ can be obtained by the following method. That is, highDO₃ is temporarily determined by extrapolation from the shape of the curve in the block, the result calculated by Equation (6) and the measurement value are compared with each other, and calculation is repeatedly performed by changing highDO₃, so that a value that most approximates to the measurement value in the block can be obtained as highDO₃. The difference between this highDOₙ and DOhf which is finally balanced when a mixed liquid in which BOD is 0 mg/l is aerated is represented by DOhf−highDOₙ=BODactn/KLa, and hence BODactn represented by Equation (7) of

BODactn=KLa×(DOhf−highDOₙ)

indicates an oxygen consumption rate when a BOD component in an n-th block is decomposed.

In general, since waste water contains a plurality of components, the above decomposition reactions are performed for respective components. FIG. 2 shows a decomposition example of waste water containing three components. For example, when waste water is composed of an X component which is easily decomposed, a Y component having a moderate decomposition properties, and a Z component having a slow decomposition rate, the block 1 indicates a process in which the X component is decomposed, the block 2 indicates a process in which the Y component is decomposed, and the block 3 indicates a process in which the Z component is decomposed. In patent Document 1, the method in which a plurality of blocks is formed is disclosed.

### Patent Document 1: Japanese Unexamined Patent Application Publication No. 2001-235462

### DISCLOSURE OF INVENTION

### Problems to be Solved by the Invention

However, in a conventional method, the decomposition rate is obtained based on the assumption in that a plurality of components contained in each block is regarded as one independent component. For example, blockₙ and blockₙ₊₁ are handled as if they are completely different components. This is an imaginary component for explaining the decomposition rate of waste water and is different from an actual component present in the waste water. This assumption is convenient to intuitionally determine whether the decomposition rate is slow or high and whether waste water is easily treated or not; however, a component decomposed in waste water and a decomposition rate thereof cannot be understood. In addition, from this analysis, actual components present in waste water cannot be identified.

Furthermore, when the decomposition rate of the conventional method is used for predictive simulation calculation of treatment conditions of activated sludge, calculation can be performed in the case in which waste water (raw water) to be treated is charged into an aeration tank only from one place located at the front portion thereof, as is the case of a standard activated sludge method, and in the case in which activated sludge has a small return sludge influence. However, for example, as in the case of step aeration, when a new raw water component is added when BOD which is not totally decomposed still remains, the oxygen consumption rate is different from that obtained based on the assumption in which a single component is only contained, and hence the calculation cannot be performed. As is the case described above, when BOD of treated waste water (treated water) is high at an outlet of an aeration tank, the amount of BOD which returns as return sludge is increased, and calculation cannot be performed since the oxygen consumption rate is different from that of the case in which raw water and activated sludge are only present. As described above, it cannot be said that the method disclosed in Patent Document 1 is satisfactory.

The present invention is to provide a highly precise analysis method of an oxygen consumption rate, which can be more widely used for aerobic microbe reaction systems.

### Means for Solving the Problems

To this end, the present invention includes the following aspects. That is, according to a first aspect, that is, claim 1, of the present invention, there is provided a method for analyzing a mixed liquid, which is used for waste water treatment using aerobic microbes, comprising the steps of: dividing a waste water treatment process into x blocks based on a step-shaped change of a dissolved oxygen-concentration change curve, the step-shaped change being formed by the difference in oxygen consumption rate of a plurality of BOD components in an aerated mixed liquid; approximating the change in dissolved oxygen concentration of each block using

DO=highDOᵢ−(highDOᵢ−DOᵢ₋₁)exp(−KLa·(t−tᵢ₋₁))(i=1˜x);

solving kᵢ assuming that the oxygen consumption rate of each block is linear combination of an oxygen consumption rate kᵢ (i=1˜x) of each BOD component contained in the block so as to obtain the oxygen consumption rate kᵢ of each BOD component; and also obtaining pBODᵢ which is a BOD concentration of each BOD component using the relationship represented by pBODᵢ=kᵢ·tₓ.

In addition, in the method described above, an oxygen consumption rate kₓ=KLa·(DOhf−highDOₓ) of a last block (block X) containing only one BOD component is first obtained by using the approximate expression of the dissolved oxygen-concentration change curve, Δtₓ=tₓ−tₓ₋₁, and BODₓ=kₓ·Δtₓ; kₓ₋₁ of a block which is second to the last block (block X-1) and which contains two BOD components is then obtained from the equation represented by kₓ₋₁=KLa·(DOhf−highDOₓ₋₁)−kₓ=KLa·(highDOₓ−highDOₓ₋₁); an oxygen consumption rate kᵢ of each BOD component is obtained as the equation represented by kᵢ=KLa·(highDOᵢ₊₁−highDOᵢ) by sequentially performing this calculation to a first block; and pBOD₁ that is the BOD concentration of each BOD component is also obtained by using the relationship represented by pBODᵢ=kᵢ·tₓ (claim 2). In this case, the total mass transfer coefficient is represented by KLa, a dissolved oxygen concentration at a time at which it reaches an approximately constant value by aeration of the mixed liquid for a sufficient long time is represented by DOhf, and the initial value of the dissolved oxygen concentration of the mixed liquid is represented by DO₀. In addition, the aeration time from the start of addition is represented by t, a dissolved oxygen concentration at the front portion of an i-th block is represented by DOᵢ₋₁, the start time is represented by tᵢ₋₁, and the completion time is represented by tᵢ. In accordance with another aspect, claim 3, of the present invention, there is provided a method for analyzing a mixed liquid, comprising the steps of: obtaining BOD of the mixed liquid at an optional position of an aeration tank by the following equation using the oxygen consumption pBODₓ and the oxygen consumption rate kᵢ of each BOD component, which are obtained as described above,

$$\begin{matrix}[ Formula 4 ] \\ {outBOD}_{i} = {pBOD}_{i} - {\int }_{0}^{tv} {k}_{i} \cdot t \cdot f ( t ) d t - {\int }_{tv}^{∞} {pBOD}_{i} \cdot f ( t ) d t\end{matrix}$$

in which a residence time distribution function is represented by f(t), and tᵥ=pBODᵢ/kᵢ is used; and a value (ΣoutBODi) obtained by integration of all components which satisfy outBODi>0 is estimated as the BOD value of the mixed liquid at the position.

The calculation method disclosed in Patent Document 1 is a calculation method in which a process is repeatedly performed such that a BOD component represented by A is present at the start point, is decomposed at an oxygen consumption rate k_{A} by decomposition, is turned into a BOD component represented by B after the decomposition, is decomposed at an oxygen consumption rate k_{B} by decomposition, is turned into a BOD component represented by C after the decomposition, and is decomposed at an oxygen consumption rate k_{c} by decomposition; the component A is finally turned into a component represented by Z in the vicinity of an outlet of the aeration tank; a non-decomposed BOD amount of the Z component by the outlet of the aeration tank is discharged as treated water; and part thereof is returned to the aeration tank as return sludge. As a result, since the BOD component of the return sludge is Z at the front portion of the aeration tank, the BOD component of raw water is different from A, and it is assumed that they are decomposed at oxygen consumption rates k_{Z} and k_{A}, respectively. When the treated water is in good conditions, since the BOD amount returned as the return sludge is small, the measurement value and the calculation value are not largely different from each other; however, when treatment is not well performed, by mixing performed in the aeration tank, the A, B, and C components are also contained in the returned sludge, and hence at the front portion of the aeration tank, the calculation is performed such that the component A in the raw water and the component A in the return sludge are respectively decomposed at the oxygen consumption rate k_{A}. However, according to this calculation, an apparent decomposition rate of the A component becomes twice an actual decomposition rate, and hence the calculation value and the measurement value may be largely different from each other in some cases. On the other hand, according to the case of the present invention, since the oxygen consumption rate by decomposition is obtained for each component, and the value thereof is used, even when the raw water is charged into the aeration tank from a plurality of positions, the calculation can be appropriately performed only by increasing amounts of components to change the component concentrations. Of course, to the case of a BOD component in which when the concentration thereof is changed, the oxygen consumption rate is changed, the method described above may not be applied; however, since the method of the present invention is based on the assumption that the oxygen consumption rate by decomposition does not depend on the concentration, in the case described above, a different model has to be used for analysis. In general, in the range of from several milligrams per liter to several hundred milligrams per liter used for operation analysis of activated sludge, even when the calculation according to the present invention is performed, the calculation result may well relate to the actual value in many cases, and hence it is understood that the analysis method of the present invention is effective.

### ADVANTAGES

By a conventional analysis method for analyzing a dissolved oxygen-concentration change curve which is obtained by adding waste water to be measured to an activated sludge mixed liquid in which BOD therein is substantially decreased to 0 mg/l by aeration of the activated sludge, estimation of the treatment state of the activated sludge cannot be sufficiently performed. On the other hand, by the analysis method according to the present invention, analysis can be performed for various cases of the activated sludge. For example, when a new treatment apparatus is built, the analysis method described above can significantly improve the efficiency of designing the apparatus. In addition, also in an existing apparatus for performing a waste water treatment, for example, the level of load for treating existing waste water can be estimated, and in addition, when new waste water is treated, the probability of the treatment and the level thereof can also be easily estimated.

### BEST MODE FOR CARRYING OUT THE INVENTION

Hereinafter, embodiments of the present invention will be described in more detail with reference to FIGS. 3 to 9. In addition, of course, the scope of the present invention is within the range of claims and is not limited to the following embodiments.

### First Embodiment

FIG. 3 is a view showing the DO change curve obtained when a solution to be measured is added under conditions in which an initial value DO₀ is DOhf. This DO change curve is divided with time into a first to an x-th block. When a dissolved oxygen concentration at the front of an n-th block, the start time and the complete time thereof are represented by DOₙ₋₁, tₙ₋₁, and tₙ, respectively, the DO change curve in the range of tₙ₋₁<t≦tₙ is approximated by Equation (10) of

DO=highDOₙ−(highDOₙ−DOₙ₋₁)exp(−KLa·(t−tₙ₋₁)

where highDOₙ is constant in each block. In addition, an oxygen consumption BOD in the n-th block is represented by BODₙ. In addition, Equation (10) is identical to the above Equation (6). The steps described above are the same as disclosed in Patent Document 1; however, according to the present invention, for individual BOD components forming BOD of raw water to be measured, the oxygen consumption rate in decomposition and the BOD amount are further calculated.

Particular calculation examples will be described. Since the DO change curve in a last x-th block corresponds to the decomposition of a last remaining BOD component, from an approximate curve of the x-th block represented by Equation (11) of

DO=highDOₓ−(highDOₓ−DOₓ−₁)exp(−KLa·(t−tₓ₋₁),

an oxygen consumption rate Kₓ in decomposition of one type of BOD component in this block is represented by Equation (12) of

Kₓ=KLa·(DOhf−highDOₓ₋₁),

and when Equation (13) of

Δtₓ=tₓ−tₓ₋₁

is used, Equation (14) of

BODₓ=kₓ·Δtₓ holds.

In this equation, BOD_{X} is the BOD concentration of the x-th block. Next, in an (x−1)-th block, since the DO change curve should correspond to the total of the decomposition of the BOD component in the x-th block and the decomposition of another BOD component, from an approximate curve of the (x−1)-th block represented by Equation (15) of

DO=highDOₓ₋₁−(highDOₓ₋₁−DOₓ₋₂)exp(−KLa·(t−tₓ₋₂)),

an oxygen consumption rate kₓ of said another BOD component in decomposition is represented by Equation (16) of

Kₓ₋₁=KLa·(DOhf−highDOₓ₋₁)−kₓ=KLa·(highDOₓ−highDOₓ₋₁),

and when Equation (17) of

Δtₓ₋₁=tₓ₋₁−tₓ₋₂

is used, BODₓ₋₁ of this block is represented by Equation (18) of

BODₓ₋₁=(kₓ₋₁+kₓ)·Δtₓ.

When calculation is sequentially performed as described above, since the DO change curve in a first block corresponds to the decomposition of all BOD components, from an approximate curve of this block represented by Equation (19) of

DO=highDO₁−(highDO₁−DO₀)exp(−KLa·t),

an oxygen consumption rate k₁ of the last BOD component in decomposition is represented by Equation (20) of

k₁=KLa·(highDO₂−highDO₁),

and when Equation (21) of

Δt₁=t₁

is used, BOD₁ of this block is represented by Equation (22) of

BOD₁=(k₁+k₂ . . . +kₓ₋₁+kₓ)·Δt₁.

As described above, kᵢ for each block can be calculated and is correlated with BODᵢ. Accordingly, by using general formulas, Equation (23) of

kᵢ=KLa·(highDOᵢ₊₁−highDOᵢ),

Equation (24) of

Δtᵢ=tᵢ−tᵢ₋₁,

### and Equation (25) of

BODᵢ=Σkᵢ·Δtᵢ

hold. Since pBODᵢ, the BOD concentration of a BOD component having an oxygen consumption rate kᵢ, is obtained by integration of the BOD concentration kᵢ·Δtᵢ in each block, and as a result, Equation (26) of

pBODᵢ=kᵢ·tᵢ

holds. FIG. 9 is a schematic view showing the relationship described above.

By using the decomposition rate data thus obtained, BOD under various treatment conditions of activated sludge can be calculated. In calculation, since “the oxygen consumption rate kᵢ by decomposition is assumed to be constant regardless of the concentration”, when a time from a calculation start position to a calculation target position is represented by t, the decomposition amount of an i-th component is represented by kᵢ·t. Hence, when a remaining BOD at the calculation target position is represented by outBODᵢ, Equation (24) of

outBODᵢ=inBODᵢ−kᵢ·t

holds. When the flow inside the aeration tank is a perfect piston flow, the residence time of the whole amount is represented by τ; however, in the actual flow in the aeration tank, the residence time has a distribution due to mixing and the like. In this case, mixing properties of the aeration tank is calculated, and a residence-time distribution is obtained, so that the decomposition amount based on the residence-time distribution is obtained for each component. Furthermore, when a remaining BODᵢ of each component is obtained by the difference from the BODᵢ at the calculation start position and is integrated for all the components, a remaining BOD can be obtained. A particular example of a BOD calculation method will be described in the case of a standard activated sludge method which is a representative activated sludge method. FIG. 4 is a view schematically showing standard activated sludge. An aeration tank generally has a long rectangular shape along the flow direction, and raw water is charged at a front portion of the aeration tank. A precipitation tank is provided at the rear side of an outlet of the aeration tank, and sludge is separated from a supernatant liquid and is then returned to the front portion of the aeration tank as return sludge.

The calculation is performed for each component. When BOD of the raw water is composed of x BOD components, the BOD concentration of an i-th component is represented by in BODᵢ, and the oxygen consumption rate thereof by decomposition is represented by kᵢ, since BOD of the return sludge is added to BOD of the raw water at the front portion of the aeration tank, pBODᵢ which is the BOD concentration of the i-th component at the front portion of the aeration tank is represented by Equation (27) of

pBODᵢ=(F·inBODᵢ+RS·(outBODᵢ−clBODᵢ))/(F+RS)

where a raw water treatment amount, a BOD concentration in the raw water, a return sludge amount, a BOD concentration of the i-th component at the outlet of the aeration tank, and a concentration change in the precipitation tank are represented by F, in BODᵢ, RS, outBODᵢ, and clBODᵢ, respectively. However, since aeration is not performed in the precipitation tank, when it is assumed that the concentration change clBODᵢ is approximately 0, Equation (28) of

pBODᵢ=(F·inBODᵢ+RS·outBODᵢ)/(F+RS)

is satisfied. When the aeration tank is composed of N perfect mixing baths having a volume of V and connected in series, as shown in FIG. 5, and the model of mixing properties is formed, a residence time distribution function (f) is represented by the following Equation (29).

$$\begin{matrix}[ Formula 5 ] & \\ f ( t ) = \frac{N}{τ} \frac{{( N θ )}^{N - 1} exp ( - N θ )}{( N - 1 ) !} & Equation ( 29 )\end{matrix}$$

In the above equation, an aeration tank volume, a flow rate, a residence time from the front portion of the aeration tank to the outlet thereof, and an average residence time are represented by NV, q, t, and τ=NV/q, respectively, so that a non-dimensional residence time is represented by θ=t/τ.

Since f(t) represents the ratio of a mixed liquid having a residence time t until the outlet of the aeration tank, the decomposition concentration of the i-th component of the mixed liquid amount (F+RS)·f(t) is represented by kᵢ×t; however, even when the component is fully decomposed, the maximum value of is pBODᵢ, and hence when tᵥ=pBODᵢ/kᵢ is used, the following equation holds.

$$\begin{matrix}[ Formula 6 ] & \\ {outBOD}_{i} = {pBOD}_{i} - {\int }_{0}^{tv} {k}_{i} \cdot t \cdot f ( t ) d t - {\int }_{tv}^{∞} {pBOD}_{i} \cdot f ( t ) d t & Equation ( 30 )\end{matrix}$$

When outBODᵢ that satisfies both Equations (28) and (30) is obtained, and integration is performed for all components which satisfy outBODᵢ>0, Equation (31) of outBOD=ΣoutBODᵢ holds. The outBOD thus obtained is a BOD value at the outlet of the aeration tank.

### Second Embodiment

Next, an example of the BOD calculation in the case of a step aeration method will be described. FIG. 6 is a view schematically showing a step aeration type activated sludge. A significant difference from the standard activated sludge treatment method is that a plurality of positions for charging raw water is provided along the flow direction. As is the case of the standard activated sludge treatment method, the model of mixing properties is formed based on the case in which the aeration tank is composed of N perfect mixing baths having a volume of V and connected in series; however, in the case of the step aeration, as shown in FIG. 6, when one position for charging raw water is provided for each perfect mixing bath, calculation can be conveniently performed. Raw water charged from different positions which are close to each other is collectively charged from one position for convenience. Calculation is performed for each perfect mixing bath. As shown in FIG. 7, the perfect mixing baths connected in series are numbered from 1 to N, and a raw water amount charged into a j-th perfect mixing bath is represented by Fⱼ. When the raw water is not charged, Fⱼ is set to 0. When the BOD concentration of an i-th BOD component charged in the j-th perfect mixing bath is represented by inBODᵢⱼ, and the BOD concentration of the i-th BOD component discharged from the j-th perfect mixing bath is represented by outBODᵢⱼ, the material balance of the first perfect mixing to which return sludge returns is represented by the following equation:

$$\begin{matrix}[ Formula 7 ] & \\ {outBOD}_{i 1} = {pBOD}_{i 1} - {\int }_{0}^{tv} {k}_{i} \cdot t \cdot f ( t ) d t - {\int }_{tv}^{∞} {pBOD}_{i 1} \cdot f ( t ) d t & Equation ( 34 )\end{matrix}$$

where Equation (33) of

pBODᵢ₁=(F₁·inBODᵢ+RS·outBODᵢₙ)/(F₁+RS),

and tᵥ=pBODᵢ₁/kᵢ are used. The material balance of the j-th perfect mixing bath except for the first mixing bath is represented by the following equation:

$$\begin{matrix}[ Formula 8 ] & \\ {outBOD}_{ij} = {pBOD}_{ij} - {\int }_{0}^{tv} {k}_{i} \cdot t \cdot f ( t ) d t - {\int }_{tv}^{∞} {pBOD}_{ij} \cdot f ( t ) d t & Equation ( 36 )\end{matrix}$$

where Equation (35) of

pBODᵢⱼ=(Fⱼ·inBODᵢⱼ+(ΣF+RS)·outBODᵢⱼ₋₁)/(ΣF+RS)

and tᵥ=pBODᵢⱼ/kᵢ are used. In the equation, t is a time required for the BOD component to pass through the j-th perfect mixing bath, and f(t) is the residence time distribution function when N is 1. In addition, ΣF is the total raw water amount and is the total sum of Fⱼ in which J is from 1 to N. Since the BOD concentration supplied to one perfect mixing bath which is other than the first perfect mixing bath and to which the raw water is not charged is the BOD concentration at the outlet of a perfect mixing bath located just before said one perfect mixing bath, z perfect mixing baths which are located in series and to which the raw water is not charged may be collectively calculated. For example, when z perfect mixing baths are sequentially provided from the j-th perfect mixing bath, the material balance of a (j+z−1)-th perfect mixing bath from the j-th mixing baths may be calculated from the following equation since pBODᵢⱼ=outBODᵢⱼ₋₁ holds:

$$\begin{matrix}[ Formula 9 ] & \\ {outBOD}_{ij + z - 1} = {pBOD}_{ij - 1} - {\int }_{0}^{tv} {k}_{i} \cdot t \cdot f ( t ) d t - {\int }_{tv}^{∞} {pBOD}_{ij} \cdot f ( t ) d t & Equation ( 37 )\end{matrix}$$

where tᵥ=pBODᵢⱼ/kᵢ is used. In the above equation, t is a time required for the BOD component to pass through the (j+z−1)-th perfect mixing bath, and f(t) is a residence time distribution function when N is z. When outBOD_{iN} that satisfies those equations is obtained and is integrated for all components which satisfy outBOD_{iN}>0, Equation (38) of

outBOD=Σout BOD_{iN}

holds. As a result, the outBOD thus obtained is a BOD value at the outlet of the aeration tank.

In the case of the step aeration, since before raw water previously charged is totally decomposed, raw water is subsequently charged, according to the method disclosed in Patent Document 1, the calculation is performed as if an A component, a B component, and a C component of the previously-charged raw water and an A component of the subsequently-charged raw water are decomposed at the same time, and hence, as a result, the A component is apparently decomposed twice faster than the actual rate. Since overlapping components are apparently decomposed at a decomposition rate obtained by multiplying the actual rate by the overlapping times, when the raw water is simply charged from a plurality of positions as described above, the decomposition amount is apparently increased by the calculation and becomes far different from the actual result. In order to avoid the case described above, in the case in which when the same component is overlapped, the component amount is increased without changing the decomposition rate, and in the case in which the same component is not overlapped, a general calculation process may be performed for all the residence time distributions. However, when the number of components is large, the number of perfect mixing baths is increased, and the raw water is charged at a plurality of positions, this calculation becomes a complicated and extremely hard work even for a computer. In addition, when the A component and the B component are simultaneously decomposed, it cannot be ensured that the actual decomposition rates are equivalent to the respective decomposition rates used for calculation. As described above, there has been a problem in that the calculation method disclosed in Patent Document 1 is difficult to be applied to a step aeration type activated sludge treatment. On the other hand, according to the calculation method of the present invention, since the oxygen consumption rate by the decomposition is obtained for each component, and the value thus obtained is used, even when the raw material is charged in the aeration tank from a plurality of positions, the calculation can be appropriately performed only by increasing each component amount so as to change each component concentration. Of course, in the case of a BOD component in which when the component concentration is changed, the oxygen consumption rate is also changed, the calculation cannot be appropriately used; however, since it is assumed that the oxygen consumption rate by decomposition does not depend on the concentration, in the case as described above, analysis must be performed based on a different model. In general, in the range of several per liter to several hundred milligrams per liter for analysis of operation conditions of activated sludge, by the method according to the present invention, the calculation well correlates with the measurement value, and hence the analysis method of the present invention can be effectively used.

Although the change amount clBODᵢ in the precipitation tank is ignored since the decomposition amount is small due to no aeration, when a large amount of nitrate ions is present in a mixed liquid at the outlet of the aeration tank, the dissolved oxygen becomes 0 mg/l in the precipitation tank, and the BOD component is consumed by a denitrification reaction caused by denitrification bacteria, so that clBODᵢ cannot be ignored. In the case described above, the denitrification reaction rate is obtained by a different method, and an effective clBODᵢ must be obtained.

### EXAMPLES

FIG. 8 is a graph showing an example of the change in dissolved oxygen concentration when waste water to be measured was added, followed by aeration, under conditions in which an initial value DO₀=DOhf was satisfied. In the figure, ◯ indicates data of dissolved oxygen and is plotted every 30 seconds. This data was divided into 4 blocks in which DOhf was 7.24 [mg/l] and KLa was 0.312[l/min], curves approximated by Equation (10) were shown by curves connected between DO0 and highDO1, DO0 and highDO2, DO2 and highDO3, and DO3 and highDO4. Furthermore, the oxygen consumption rate of each BOD component, its BOD, and the ratio thereof to the total BOD obtained from Equations (23) to (25) are shown in Table 1. The oxygen consumption rate of each BOD component, its BOD, and the ratio to the total BOD analyzed by the method disclosed in Patent Document 1 are shown in Table 2 for reference.

TABLE 1

| | BOD Component No. | | | |
|---|---|---|---|---|
| | Com. 1 | Com. 2 | Com. 3 | Com. 4 |
| Oxygen consumption rate by | 0.299 | 0.276 | 0.055 | 0.041 |
| decomposition [mg/l/min] | | | | |
| BOD Concentration [mg/l] | 3.50 | 1.24 | 1.05 | 1.48 |
| Ratio to Total BOD [%] | 48.2 | 17.1 | 14.4 | 20.3 |

TABLE 2

| | BOD Component No. | | | |
|---|---|---|---|---|
| | Com. 1 | Com. 2 | Com. 3 | Com. 4 |
| Oxygen consumption rate by | 0.67 | 0.40 | 0.096 | 0.041 |
| decomposition [mg/l/min] | | | | |
| BOD Concentration [mg/l] | 3.02 | 2.88 | 0.71 | 0.65 |
| Ratio to Total BOD [%] | 41.6 | 39.6 | 9.8 | 9.0 |

Next, with reference to Table 3, a calculation example will be described which was applied to an actual standard activated sludge treatment apparatus using the analysis data shown in Table 1. Under conditions in which the volume of an aeration tank was 630 m³, the volume of raw water for treatment was 8 m³/hr, and the volume of return sludge was 8 m³/hr, the BOD concentration of the raw water was changed from 600 to 1,400 mg/l, the mixing properties of the aeration tank was approximated by the model formed on the case in which 4 perfect mixing baths were connected in series, and BOD of the treated water was obtained from Equations 28 to 31 and is shown in the fourth line of the table. For comparison purposes, BOD of the treated water calculated in accordance with the method disclosed in Patent Document 1 is shown in the fifth line of the table, the calculation being performed using the data shown in Table 2 obtained by the analysis method disclosed in the above document. By the comparison of data between the fourth and the fifth lines, it is understood that when the concentration of the raw water is low, a significant difference cannot be observed between the two calculation methods; however, when the concentration of the raw water is increased, and BOD of the treated water is increased, the influence of return sludge is increased, so that a large difference can be observed. In addition, in the sixth line of the table, measured data of BOD of treated water is shown which was obtained by a small activated sludge test machine in which the activated sludge was set to a scale of 1/630,000. In addition, in the seventh line, measured data of BOD of the treated water is shown which was obtained when the BOD concentration of the raw water of the above activated sludge was 1,200 mg/l. The results shown in the sixth and the seventh lines are close to the results by the calculation method of the present invention and show that this calculation method is effective.

TABLE 3

| | Calculation No. | | | | | | |
|---|---|---|---|---|---|---|---|
| | No. 1 | No. 2 | No. 3 | No. 4 | No. 5 | No. 6 | No. 7 |
| BOD in raw water [mg/l] | 600 | 700 | 800 | 900 | 1000 | 1200 | 1400 |
| Volume load of BOD [Kg/m3/day] | 0.18 | 0.21 | 0.24 | 0.27 | 0.30 | 0.37 | 0.43 |
| Calculation result of treated | 5.7 | 10.4 | 17.2 | 26.9 | 39.0 | 73.7 | 120 |
| water BOD by data in Table 1 | | | | | | | |
| [mg/l] | | | | | | | |
| Calculation result of treated | 4.7 | 8.2 | 13.0 | 19.2 | 26.9 | 46.6 | 72.1 |
| water BOD by data in Table 2 | | | | | | | |
| [mg/l] | | | | | | | |
| Measured result of treated | | 10 | | 26 | | 70 | |
| water BOD by small activated | | | | | | | |
| sludge test machine [mg/l] | | | | | | | |
| Measured result of treated | | | | | | 72 | |
| water BOD by actual activated | | | | | | | |
| sludge [mg/l] | | | | | | | |

### BRIEF DESCRIPTION OF THE DRAWINGS

FIG. 1 is a view for illustrating the principle according to the present invention.

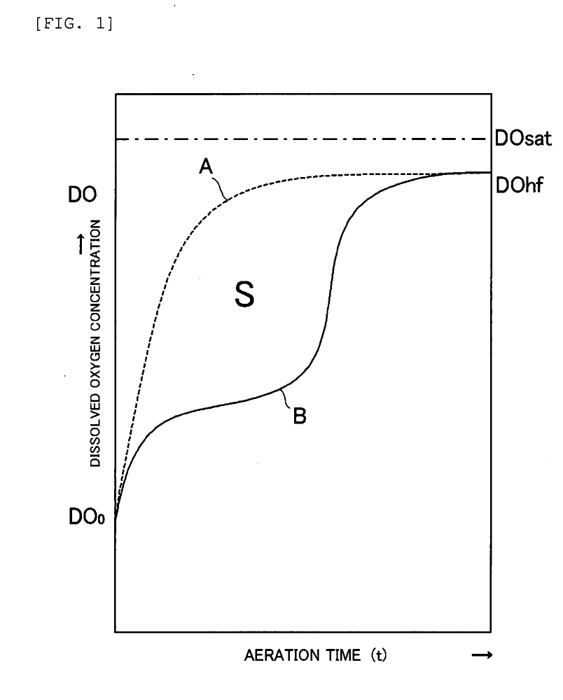

FIG. 2 is a view for illustrating a method for obtaining a BOD decomposition rate according to a conventional method.

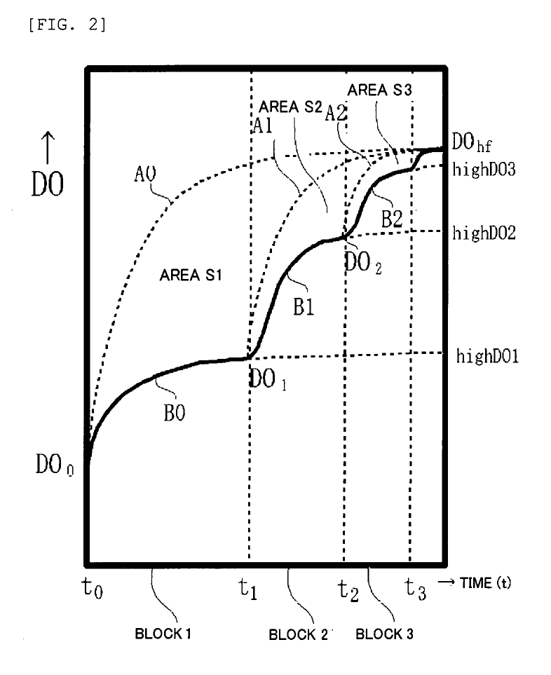

FIG. 3 is a view for illustrating a method for obtaining a BOD decomposition rate according to the present invention.

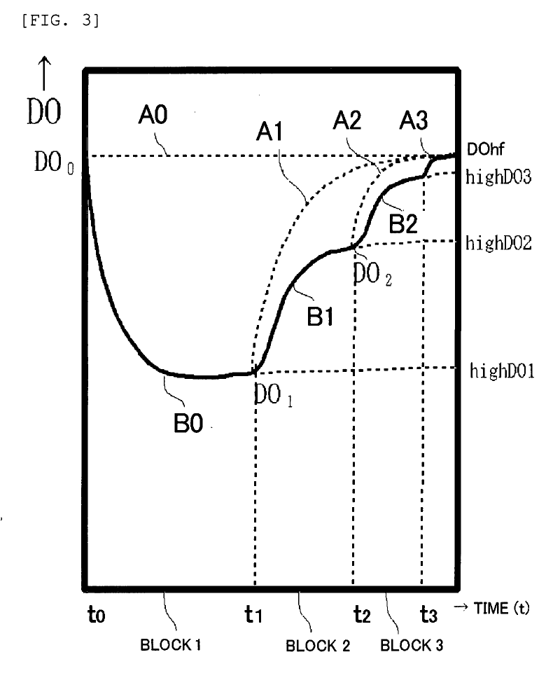

FIG. 4 is a view for illustrating a standard activated sludge method.

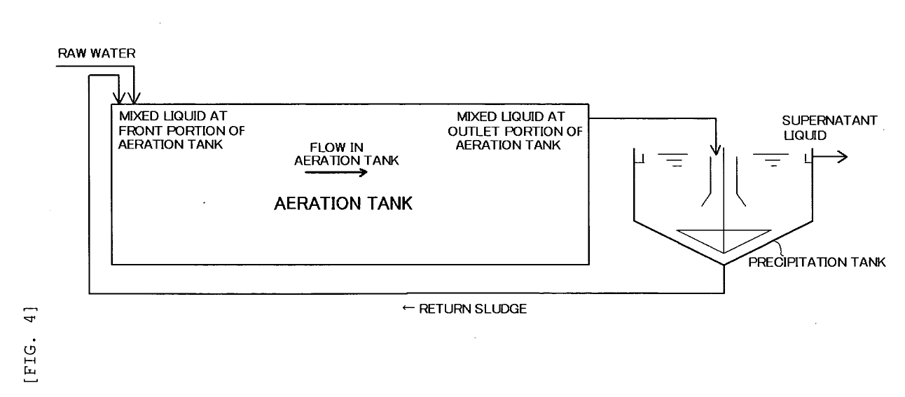

FIG. 5 is a view for illustrating a method for analyzing a standard activated sludge method.

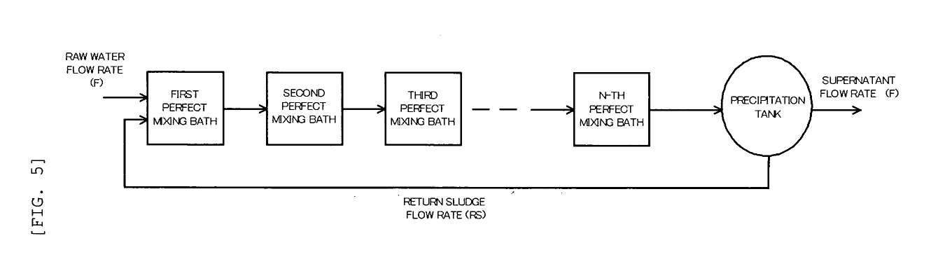

FIG. 6 is a view for illustrating a step aeration method.

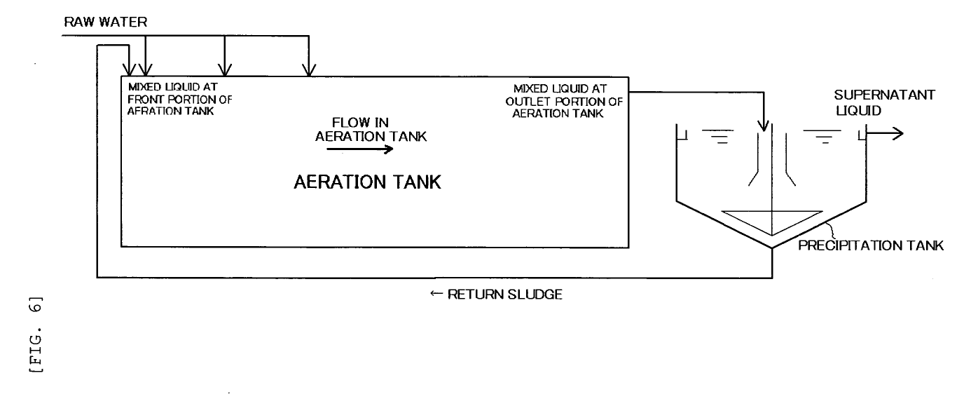

FIG. 7 is a view for illustrating a method for analyzing a step aeration method.

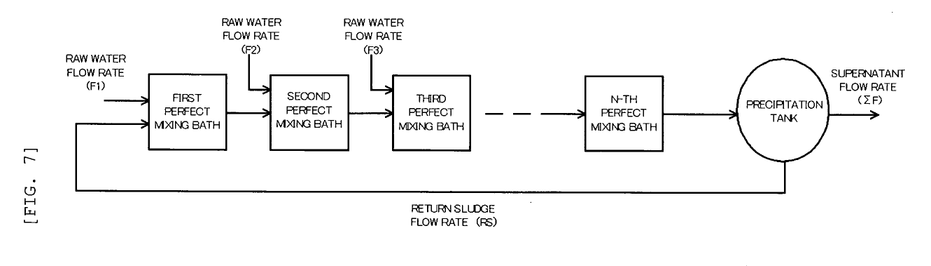

FIG. 8 is a graph showing the relationship between the decomposition rate and measured data of the change in dissolved oxygen concentration.

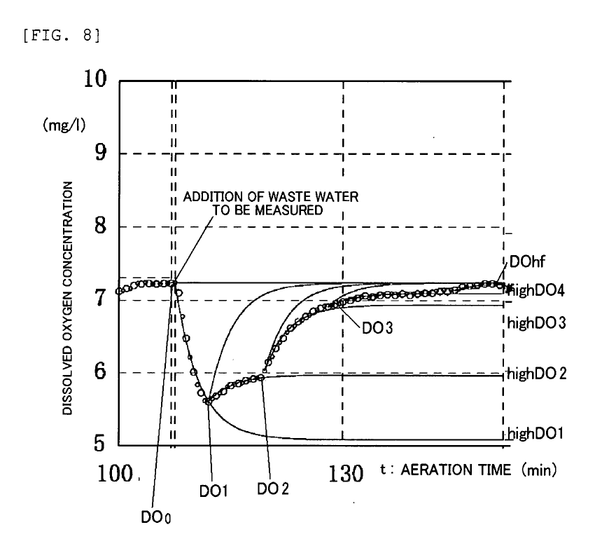

FIG. 9 is a view schematically showing calculation results according to the present invention.

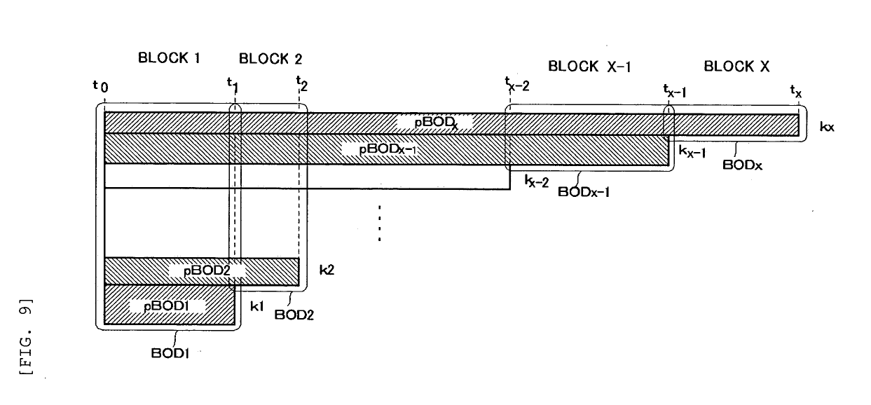

1. A method for analyzing a mixed liquid, which is used for waste water treatment using aerobic microbes, comprising the steps of:
dividing a waste water treatment process into x blocks based on a step-shaped change of a dissolved oxygen-concentration change curve, the step-shaped change being formed by the difference in oxygen consumption rate of a plurality of BOD components in an aerated mixed liquid;
approximating the change in dissolved oxygen concentration of each block using
DO=highDOᵢ−(highDOᵢ−DOᵢ₋₁)exp(−KLa·(t−tᵢ₋₁))(i=1=1˜x);
solving kᵢ assuming that the oxygen consumption rate of each block is linear combination of an oxygen consumption rate kᵢ(i=1˜x) of each BOD component contained in the block so as to obtain the oxygen consumption rate kᵢ of each BOD component;
and also obtaining pBODᵢ which is a BOD concentration of each BOD component using the relationship represented by pBODᵢ=kᵢ−tᵢ.

2. The method for analyzing a mixed liquid, according to claim 1, wherein an oxygen consumption rate
kₓ=KLa·(DOhf−highDOₓ) of a last block (block X) containing only one BOD component is first obtained by using the approximate expression of the dissolved oxygen-concentration change curve, Δtₓ=tₓ−tₓ₋₁, and BODₓ=kₓΔtₓ;
kₓ₋₁ of a block which is second to the last block (block X−1) and which contains two BOD components is then obtained from the equation represented by
kₓ₋₁=KLa·(DOhf−highDOₓ₋₁)−kₓ=KLa·(highDOₓ−highDOₓ₋₁);
an oxygen consumption rate kᵢ of each BOD component is obtained as the equation represented by
kᵢ=KLa·(highDOᵢ₊₁−highDOᵢ) by sequentially performing this calculation to a first block;
and pBODᵢ that is the BOD concentration of each BOD component is obtained by using the relationship represented by pBODᵢ=kᵢ·tᵢ

3. A method for analyzing a mixed liquid, comprising the steps of:
obtaining BOD of the mixed liquid at an optional position in an aeration tank by the following equation using the oxygen consumption pBODₓ and the oxygen consumption rate kᵢ of each BOD component, which are obtained according to claim 1,
$$\begin{matrix}[ Formula 1 ] \\ {outBOD}_{i} = {pBOD}_{i} - {\int }_{0}^{tv} {k}_{i} \cdot t \cdot f ( t ) d t - {\int }_{tv}^{∞} {pBOD}_{i} \cdot f ( t ) d t\end{matrix}$$
in which a residence time distribution function is represented by f(t), and tᵥ=pBODᵢ/kᵢ holds;
and a value (ΣoutBODi) obtained by integration of all components which satisfy outBODi>0 is estimated as the BOD value of the mixed liquid at the position.

4. A method for analyzing a mixed liquid, comprising the steps of:
obtaining BOD of the mixed liquid at an optional position in an aeration tank by the following equation using the oxygen consumption pBODₓ and the oxygen consumption rate kᵢ of each BOD component, which are obtained according to claim 2,
$$\begin{matrix}[ Formula 1 ] \\ {outBOD}_{i} = {pBOD}_{i} - {\int }_{0}^{tv} {k}_{i} \cdot t \cdot f ( t ) d t - {\int }_{tv}^{∞} {pBOD}_{i} \cdot f ( t ) d t\end{matrix}$$
in which a residence time distribution function is represented by f(t), and tᵥ=pBODᵢ/kᵢ holds;
and a value (ΣoutBODi) obtained by integration of all components which satisfy outBODi>0 is estimated as the BOD value of the mixed liquid at the position.
